(19) United States
(12) Patent Application Publication
(10) Pub. No.: US 2024/0041778 A1
(43) Pub. Date: Feb. 8, 2024

(54) CONCENTRATED LIQUID GEL FORMULATIONS CONTAINING NAPROXEN SALTS

(71) Applicant: Bayer Healthcare LLC, Whippany, NJ (US)

(72) Inventors: Blanca ALVAREZ MALUENDA, Madrid (ES); Maria Elena IGLESIAS PINEIRO, Alcalá de Henares (ES); Maria Pilar SANZ SAIZ, Madrid (ES); Anthony EKPE, Parlin, NJ (US)

(73) Assignee: Bayer Healthcare LLC, Whippany, NJ (US)

(21) Appl. No.: 18/232,302

(22) Filed: Aug. 9, 2023

Related U.S. Application Data
(63) Continuation of application No. 18/271,334, filed on Jul. 7, 2023.
(60) Provisional application No. 63/135,885, filed on Jan. 11, 2021.

(51) Int. Cl. A61K 9/48; A61K 31/192; A61K 47/02; A61K 47/10; A61K 47/32

(52) U.S. Cl. CPC A61K 9/4825; A61K 31/192; A61K 47/02; A61K 47/10; A61K 47/32

(57) ABSTRACT

This disclosure is in the field of mini-softgel capsules, particularly softgel capsules containing naproxen salt as an active ingredient. It relates generally to softgel capsules containing high concentration formulations of naproxen sodium.

### CROSS-REFERENCE TO RELATED APPLICATIONS

This application claims the benefit of U.S. Provisional Application No. 63/135,885, filed on Jan. 11, 2021, the entire contents of which are incorporated herein by reference.

### BACKGROUND OF THE INVENTION

Soft gelatin capsules (“softgels” or “soft gels”) are used to enclose a variety of ingredients, including pharmaceuticals, in an outer shell material that seals a gelatinous fill material within and separates the fill material from the exterior environment. Softgels are useful for the oral delivery of various pharmaceutically active ingredients to patients, and soft gel formulations can provide certain advantages over other oral dosage forms. For example, softgel capsules are easier to swallow than hard capsules; they digest quickly in the gastrointestinal tract (“GI”); and they are aesthetically pleasing. Softgels can be used for delivery of a wide variety of ingredients, such as, vitamins, nutritional supplements, and active pharmaceutical ingredients (“APIs”). One preferred fill formulation for softgels is a liquid fill.

Not all liquids are suitable for softgel capsules, however. Water tends to dissolve the gelatin shell; therefore, liquids that contain water greater than about 20% by weight are generally unsuitable. Other solvents typically used in pharmaceutical formulations, such as glycerin, propylene glycol, ketones, alcohols, acids, amines, and esters all have a tendency to undermine the integrity of the gelatin shell. One significant limitation to the softgel liquid fills is their pH value. At pH values below 2.5, gelatin is hydrolyzed causing leakage of the soft gelatin capsule, whereas at pH values above 7.5, gelatin may be either hydrolyzed or tanned (i.e., cross-linked) resulting in decreased solubility of the gelatin shell.

There are also significant size limitations posed by the use of certain APIs in softgel formulations. Many APIs require solvent volumes that are simply too large for use in a softgel small enough for oral ingestion, often due to their insolubility. Either large amounts of solvent are required for solvation, or the solutions have characteristics that tend to degrade, hydrolyze, or discolor the gelatin shell. Some references have disclosed solutions for enhancing the solubility of such APIs. U.S. Pat. Nos. 5,071,643 and 5,360,615, for example, disclose systems for increasing the solubility of certain APIs through the use of hydroxide ion species. U.S. Pat. No. 6,387,400 discloses the use of a combination of hydroxide ions and polyethylene glycol that utilizes the incremental addition of hydroxide ions to solubilize the API. U.S. Pat. No. 6,689,382 also discloses the use of hydroxide ions to increase the solubility of certain insoluble, acidic APIs. U.S. Pat. No. 6,383,515, meanwhile, discloses a solution that combines polyethylene glycol and acid salts to increase API solubility. These references disclose methods that rely on converting an API from the free acid or base to its corresponding salt form. One problem with these methods is the tendency of the anions formed from acidic APIs to generate polyethylene glycol esters and reduce the amount of free pharmaceutical agent.

Naproxen is a particularly problematic API for use in softgel formulations; it is essentially insoluble in water and has limited solubility in the excipients that are typically used with softgel formulations, such as polyethylene glycol. Combining the acid form of naproxen (“naproxen acid”) with hydroxide ions to neutralize the acid results in large volumes of fill material, which is incompatible with small softgels that contain concentrated solutions of naproxen. And combining naproxen salts, such as naproxen sodium, with polyethylene glycol generates an alkaline solution that degrades the softgel shell. Such methods are, therefore, unsuited for making small softgels containing concentrated formulations of naproxen.

The above references and others disclose softgel fill materials that increase the fill volume, and require multiple manufacturing process steps, which concomitantly increases manufacturing costs. U.S. Pat. No. 10,463,637, for example, discloses formulations that combine: naproxen acid, naproxen sodium, polyethylene glycol, propylene glycol, povidone, and water, and the formulations require balancing the amounts of naproxen and naproxen sodium. Similarly, U.S. Pat. No. 9,693,979 discloses formulations that combine: naproxen sodium, lactic acid, polyethylene glycol, and polyvinylpyrrolidone, propylene glycol and a neutralizing agent that can include citric acid, malic acid, acetic acid, propionic acid, pyruvic acid, butanoic acid, and lactic acid.

There is a need, therefore, for concentrated naproxen fill compositions that avoid the requirement of combining naproxen acid with large volumes of hydroxide ions or naproxen salts with large volumes of acid to generate naproxen formulations that are suitable for use in smaller softgel capsules, include a minimal amount of excipients, and require fewer manufacturing steps.

### SUMMARY OF THE DISCLOSURE

The present disclosure is generally related to softgel capsule fill materials and to softgel capsules containing concentrated naproxen sodium formulations, wherein: each capsule contains 220 mg of naproxen sodium; the naproxen sodium is fully dissolved within; the capsules are smaller in size than currently available formulations; they contain a minimal amount of excipients; and the formulations do not react with the softgel shell material. The present disclosure provides these characteristics through softgel fill formulations that contain naproxen sodium solubilized in a limited number of excipients and encapsulated in gelatin capsules.

The present disclosure solves problems of prior technologies by providing softgel capsule compositions containing concentrated salt forms of naproxen in combination with certain excipients that are suitable for inclusion in smaller volumes.

### DEFINITIONS

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular.

As used herein, the terms “comprising” (and any form of comprising, such as “comprise,” “comprises,” and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, the term “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods. When “about” is present before a series of numbers or a range, it is understood that “about” can modify each of the numbers in the series or range.

As used herein, the terms “active agent” “active ingredient,” “active pharmaceutical ingredient,” “API,” or “drug” refer to any compound intended to alter a physical condition or state or to produce a therapeutic, prophylactic, or other intended effect. With respect to specific active agents, these terms include the pharmaceutically active agents and all pharmaceutically acceptable salts, stereoisomers, crystalline forms, complexes, cocrystals, solutions, esters, ether, hydrates, solvates, and mixtures thereof, where a particular form is pharmaceutically active.

The term “naproxen sodium” refers to the sodium salt form of naproxen, a member of the arylacetic acid group of non-steroidal anti-inflammatory drugs (NSAIDs) with anti-inflammatory analgesic and antipyretic properties. Naproxen sodium reversibly and competitively inhibits cyclooxygenases (COX), thereby blocking the conversion of arachidonic acid to pro-inflammatory prostaglandins. Its chemical names include: (S)-2-Naphthalene acetic acid, 6-methoxy-α-methyl-, sodium salt; (S)-(-)-S odium-6-methoxy-α-methyl-2-naphthalene acetate; and Sodium (2 S)-2-(6-methoxynaphthalen-2-yl) propanoate.

The term “mini-soft gel” or “mini-gel” refers to a softgel capsule that has a capsule size of NMT 8-14 minims The size and shape of such a mini-gel can vary and may include, for example, spherical, oval, oblong, or other shape.

The term “concentrated softgel fill” or “concentrated fill” refers to a softgel fill formulation containing 220 mg of naproxen sodium where the concentration of naproxen sodium is at least 28% by weight. It also refers to a softgel fill formulation containing 220 mg of naproxen sodium contained within a softgel capsule that is 14 minims or less in size. It could also refer to a softgel fill formulation containing 220 mg of naproxen sodium solubilized in a liquid fill mixture that is less than or equal to 620 microliters in total volume.

### DETAILED DESCRIPTION

In the following description, reference is made to certain formulations and specific embodiments that form a part hereof. The illustrative embodiments described in the detailed description and claims are not meant to be limiting. Other embodiments may be utilized, and other changes may be made, without departing from the scope of the present subject matter. Aspects of the present disclosure, can be arranged, substituted, combined, separated, and designed in a wide variety of different configurations, all of which are contemplated herein.

References in the specification to “one embodiment”, “an embodiment”, “an example embodiment” or “some embodiments,” etc. indicate that the embodiments described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be achieved in connection with other embodiments whether or not explicitly described.

The embodiments of the present disclosure relate to softgel capsules containing a liquid fill that includes naproxen salt, an ionizing agent, and certain excipients. In certain embodiments, the combination of ionizing agent and naproxen salt in the liquid fill will equilibrate to generate the acid form of naproxen in the fill solution. The equilibrium state of naproxen can vary depending on the particular embodiment, excipients, manufacturing methods, humidity, storage conditions, and other factors. A person of ordinary skill would understand that such variations in equilibrium state of the naproxen salt are encompassed by the present disclosures.

Embodiments of mini-gels can include different sizes. In some embodiments, the softgel capsule sizes may range from 8 to 14 minims, from 10 to 12 minims, and all sizes in between, including, for example, 8, 8.5, 9, 10, 11, 12, 13, or 14. The mini-gels can also vary in shape; they can be oblong, ovals, spheres, or other shapes. In certain embodiments, the mini-gels may be about 14% to about 30% smaller than an existing equivalent dosage form (e.g., about 14% to about 30% smaller than Aleve® Liquid-Gel Naproxen Sodium 220 mg (“Aleve®”).

The inventors faced numerous problems in the development of miniature softgel capsules containing naproxen salt. First, it is necessary to maintain the pH of the softgel fill material within the range of 2.5 to 7.5 to prevent shell dissolution at the lower end and shell hardening and discoloration at the higher end. Second, there is a need to limit the number and amounts of different excipients included in the softgel fill. Naproxen is a hydrophobic API, which needs to be solubilized in a non-aqueous solution. The typical excipients for solubilization of hydrophobic APIs such as naproxen are polyethylene glycol, glycerine, propylyne glycol, povidone or copovidone, diethylene glycol monoethyl ether, and poloxamer 124. The use of multiple different excipients increases fill volume, which is incompatible with the mini-softgel of the present disclosure. And third, some excipients are incompatible with naproxen in mini-softgels and form suspensions, precipitates, or non-flowable liquids during manufacturing.

To reduce the volume of the fill in the formulation, the inventors focused on formulations containing naproxen salts, including naproxen sodium. Naproxen sodium is the sodium salt form of naproxen, a member of the arylacetic acid group of non-steroidal anti-inflammatory drugs (NSAIDs) with anti-inflammatory analgesic and antipyretic properties. Naproxen sodium is a COX inhibitor, thereby blocking the conversion of arachidonic acid to pro-inflammatory prostaglandins. This inhibits the formation of prostaglandins that are involved in pain, inflammation and fever. Solutions containing naproxen sodium are alkaline, which is corrosive to the soft gelatin shell. To reduce the pH and the correspondent corrosive effect, it is necessary to use a neutralizing agent or acid. But the inventors surprisingly discovered that not all acids are equally useful in mini-softgel fill formulations.

To maintain softgel integrity and prevent degradation, the pH of the softgel fill must be maintained between a pH of 2.5 and 7.5. In addition, an Aleve® capsule weighs approximately 1,358 mg, with a length of 20.83 mm and a diameter of 9.36 mm (for oblong capsules), while the capsules of some embodiments in the present disclosure weigh less than about 1,100 mg per capsule, and a length of 15.34 mm and diameter of 10.25 mm (oval).

Softgel capsule shells have also been disclosed previously. Below are some examples.

| | % by | % by | % by | % by | % by |
|---|---|---|---|---|---|
| Material | Weight | Weight | Weight | Weight | Weight |
| Gelatin | 26 | 26 | 24 | 26 | 25 |
| Glycerol | 35 | 35 | 40 | 36 | 24 |
| Water | 27 | 22 | 20 | 22 | 23 |
| Starch Acetate | 12 | 17 | 16 | 16 | 16 |
| Sorbitol | 0 | 0 | 0 | 0 | 12 |
| Total | 100 | 100 | 100 | 100 | 100 |

### EXAMPLES

The below examples provide specific embodiments. The specific embodiments show exemplary capsules that can be made according to the teachings contained herein, but the use of these specific examples is not intended to be limiting.

The representative embodiments below provide high concentration naproxen sodium fill formulations that contain phosphoric acid. In addition to the below disclosures, additional embodiments exclude the following acids: fumaric acid, maleic acid, tartaric acid, citric acid, malic acid, acetic acid, propionic acid, pyruvic acid, butanoic acid, and lactic acid.

### Example 1

Representative Embodiment of High Concentration Naproxen Softgel Fill Formulations: The inventors have developed two examples of high concentration formulations that met the requirements of a mini-softgel (a reduction in size of at least 14% as compared to Aleve®, maintaining a pH for the fill solution of between about 2.5 and 7.5, and eliminating propylene glycol and glycerine from the fill formulation). One is provided below.

### Softgel Fill Formulation

| | Formulation 1 |
|---|---|
| Ingredient | (Mg/cp) |
| Naproxen Sodium | 220.00 |
| Polyethylene Glycol 400 | 435.00 |
| Copovidone | 20.00 |
| Phosphoric Acid (85%) | 33.50 |
| Total | 708.50 |
| Weight reduction | 20% |

### Capsule Formulation

| Ingredient | Percentage |
|---|---|
| Gelatin | 45.00 |
| Sorbitol sorbitan solution | 12.00 |
| Glycerol | 12.00 |
| FD&C Blue #1 | 0.020 |
| Candurin Silver Lustre | 0.500 |
| Purified water | 30.48 |
| Total | 100.00 |

### Stability Testing

Capsules containing Formulation 1 were physically stable for 6 months in the following storage conditions: 25° C.-60% RH; 30° C./65% RH; and 40° C.-75% RH. Capsule brittleness was observed in samples stored at 40° C.-75% RH; therefore, the inventors reduced the percentage of glycerol from 12% to 10% and accordingly increasing the sorbitol from 12% to 14% in the capsule shell.

The results at 6 months are provided below for both and bottle storage and blister pack for the different conditions.

### Bottle Storage Testing of Formulation 1 at 6 Months

| | | | |
|---|---|---|---|
| | 25° C.- | 30° C.- | 40° C.- |
| Parameter | 60% RH | 65% RH | 75% RH |

Assay (%)

| | | | |
|---|---|---|---|
| Naproxen sodium | 96.0 | 95.4 | 92.7 |

Dissolution test (%)
USP <711>

| | | | |
|---|---|---|---|
| Naproxen sodium | 98 | 98 | 96 |

Impurities/Degradation products (%)

| | | | |
|---|---|---|---|
| PEG esters | 2.3 | 3.2 | 6.1 |
| SORB esters | n.d. | 0.1 | 0.2 |
| GLY esters | 0.4 | 0.6 | 1.4 |
| Single Unspecified | n.d. | n.d. | n.d. |
| Total degradants | n.d. | n.d. | n.d. |
| (no esters included) | | | |

### Blister Pack Testing of Formulation 1 at 6 Months

| | | | |
|---|---|---|---|
| | 25° C.- | 30° C.- | 40° C.- |
| Parameter | 60% RH | 65% RH | 75% RH |

Assay (%)

| | | | |
|---|---|---|---|
| Naproxen sodium | 96.8 | 94.9 | 93.8 |

Dissolution test (%)
USP <711>

| | | | |
|---|---|---|---|
| Naproxen sodium | 97 | 98 | 95 |

Impurities/Degradation products (%)

| | | | |
|---|---|---|---|
| PEG esters | 2.2 | 3.1 | 6.2 |
| SORB esters | n.d. | 0.1 | 0.2 |
| GLY esters | 0.4 | 0.6 | 1.3 |
| Single Unspecified | n.d. | n.d. | n.d |
| Total degradants | n.d. | n.d. | n.d. |
| (no esters included) | | | |

### Example 2

### Softgel Fill Formulation

| | Formulation 2 |
|---|---|
| Ingredient | (Mg/cp) |
| Naproxen Sodium | 220.00 |
| Miglyol 812 | 300.00 |
| Beeswax | 10.00 |
| Soya lecithin | 10.00 |
| Total | 540.00 |
| Weight reduction | >35% |

### Capsule Formulation

| Ingredient | Percentage |
|---|---|
| Gelatin | 45.00 |
| Sorbitol-sorbitan solution | 12.00 |
| Gycerol | 12.00 |
| FD&C Blue #1 | 0.020 |
| Candurin Silver Lustre | 0.500 |
| Purified water | 30.48 |
| Total | 100.00 |

### Stability Testing

Stability testing of capsules containing Formulation 2 were conducted. Capsules containing Formulation 2 were physically stable for 6 months in the storage conditions of 25° C.-60% RH and 30° C./65% RH. For capsules maintained at 40° C.-75% RH, the inventors observed moderate sticking and rugosity in the capsule surface. In addition, one leaking capsule due to a pore in the seam was observed at 3 months.

For Formulation 2, the inventors observed a high level of brittleness of the capsules. In addition, dissolution testing showed that it would not be suitable for immediate release formulations

| | Dissolution Test USP |
|---|---|
| | <711> + Pancreatin |
| | (75 RPM) |
| Dissolution Time | Naproxen sodium (%) |
| Naproxen Sodium (15 min) | 5 |
| Naproxen Sodium (30 min) | 10 |
| Naproxen Sodium (45 min) | 12 |
| Naproxen Sodium (1 hour) | 13 |
| Naproxen Sodium (2 hours) | 17 |
| Naproxen Sodium (3 hours) | 20 |
| Naproxen Sodium (4 hours) | 23 |
| Naproxen Sodium (5 hours) | 25 |
| Naproxen Sodium (6 hours) | 27 |
| Naproxen Sodium (24 hours) | 52 |

### Example 3

Commercially Available Naproxen Formulation: Softgels containing naproxen sodium are sold commercially. Aleve® is one example. The goal was to create minigels containing concentrated naproxen sodium solutions, and Aleve® softgels do not meet these requirements.

### Example 4

Fill Formulations Containing Pivalic Acid: The inventors also discovered that fill formulations containing pivalic acid, for example, are unsuitable for use in softgel fill material because it is not included in the FDA Inactive Ingredients for Approved Drug Products.

### Example 5

Fill Formulations Containing Propionic Acid: The inventors tested formulations containing propionic acid and found that there was a limited range of propionic acid that could be used in the softgel fill to meet the requirements of a concentrated solution (between 0.69 and 2.29 mol- equivalents of propionic acid/mol-equivalents of naproxen sodium.) During stability testing, however, the inventors determined that when encapsulated, the fill formulations generated unacceptable levels of glycerol esters at mol-equivalents propionic acid/molar equivalents of naproxen sodium that were above 0.71. To prevent formation of such esters, the molar equivalent of propionic acid had to be reduced to unacceptable levels that resulted in a capsule fill pH that is above 7.5, which lies outside the required range of 2.5 to 7.5.

### Example 6

Fill Formulations Containing Phosphoric Acid: The inventors discovered, however, that phosphoric acid could be used to neutralize naproxen sodium and achieve pH levels for the fill solution that lay within the desired range, but phosphoric acid posed additional, significant barriers to creating the concentrated softgel fill of some embodiments. An important aspect of the present disclosure is maintaining a specific range of molar equivalents of acid to naproxen salt. The inventors found that above 1.5 molar equivalents of acid to naproxen salt, there was phase separation due the presence of insoluble substances. In addition, at molar equivalents of 1.2, there was some interaction with certain excipients when the solution was incubated at 40° C. They found that there was a narrow range in which phosphoric acid could be used as a neutralizing agent for naproxen sodium.

| | | Molar | Flow | Viscosity | Density | | |
|---|---|---|---|---|---|---|---|
| Trial | Copovidone | Equivalent* | Test | cPs | (g/mL) | pH | Compatibility |
| 1 | 20 | 0.8 | 177 | 1204 | 1.179 | 7.55 | Yes |
| 2 | 20 | 1.0 | 140 | 877.8 | 1.187 | 6.99 | Yes |
| 3 | 20 | 1.2 | 143 | 927.8 | 1.184 | 6.74 | No |
| 4 | 40 | 0.8 | 247 | 1668 | 1.182 | 7.69 | Yes |
| 5 | 40 | 1.0 | 214 | 1334 | 1.181 | 7.20 | Yes |
| 6 | 40 | 1.2 | 200 | 1325 | 1.185 | 6.80 | No |

*“Molar equivalents” refers to the molar equivalents of phosphoric acid per mole of naproxen sodium. In addition, the inventors discovered that at 1.5 molar equivalents of acid to naproxen sodium, there was a phase separation due to the presence of insoluble substances.

### Example 7

Excipient Compatibility with Phosphoric Acid: Not all excipients are compatible with concentrated fill solutions containing naproxen sodium. For example, the inventors discovered that polyethylene-polypropylene glycol (polaxamer 124) is incompatible with fill solutions containing naproxen sodium. It formed precipitates in a large percentage of the solutions containing naproxen sodium. The inventors also discovered that it was necessary to eliminate the use of propylene glycol as a solubilizer in the concentrated fill formulation of some embodiments of the present disclosure. The inventors found that polypropylene glycol generated brittle shells through plasticization of the shell material.

Phosphoric acid (85% solution) showed an adverse interaction with povidone and copovidone when naproxen sodium was present in the fill, creating waxy precipitates formed of insoluble orthophosphates. Povidone and copovidone play important roles in many softgel formulations by inhibiting crystallization of API and enhancing API release in an aqueous environment. The addition of alkali excipients, glycerin, or propylene glycol did not ameliorate the problem. The inventors found it necessary to use copovidone, rather than povidone in some embodiments of the present disclosure.

Numerous other excipients were incompatible with a concentrated naproxen fill formulation as shown below.

| | Mg/cp | Mg/cp | Mg/cp | Mg/cp | Mg/cp | Mg/cp | Mg/cp | Mg/cp |
|---|---|---|---|---|---|---|---|---|
| Ingredient | | | | | | | | |
| Naproxen Sodium | 220.0 | 220.0 | 220.0 | 220.0 | 220.0 | 220.0 | 220.0 | 220.0 |
| Diethylene glycol | 200.0 | 200.0 | 200.0 | 200.0 | 0 | 0 | 0 | 0 |
| mono-ethyl ether | | | | | | | | |
| Polaxamer 124 | 0 | 0 | 0 | 0 | 550.0 | 550.0 | 550.0 | 550.0 |
| Copovidone | 20.0 | 40.0 | 20.0 | 40.0 | 20.0 | 40.0 | 20.0 | 40.0 |
| Glycerol | 20.0 | 20.0 | 0 | 0 | 20.0 | 20.0 | 0 | 0 |
| Phosphoric Acid | 33.5 | 33.5 | 33.5 | 33.5 | 33.5 | 33.5 | 33.5 | 33.5 |
| (85%) | | | | | | | | |
| Total | | | | | | | | |
| % Weight reduction | 43.9 | 41.6 | 46.2 | 43.9 | 4.1 | 1.9 | 6.4 | 4.1 |

Formulations containing polaxamer 124 resulted in high viscosity suspensions, while those containing diethylene glycol monoethyl ether formed phase separations. Formulations containing polyethylene glycol, meanwhile, did not show any of these adverse reactions, but the inventors needed to discover a way to reduce the amount required to solubilize naproxen sodium.

### Example 8

Reducing Excipient Volume in Phosphoric Acid Formulations: To reduce the amount of added excipients, the inventors tested several formulations containing polyethylene glycol and varying the acid amounts (between 0.9 and 1.0 mol-equivalents of acid/mol equivalents of naproxen sodium, as shown in the below table.

| Ingredient | Mg/cp | Mg/cp | Mg/cp | Mg/cp |
|---|---|---|---|---|
| Naproxen Sodium | 220.0 | 220.0 | 220.0 | 220.0 |
| Polyethylene Glycol 400 | 300.00 | 300.00 | 300.00 | 200.0 |
| Copovidone | 12.00 | 12.00 | 0 | 0 |
| Povidone K90 | 0 | 0 | 2.00 | 2.00 |
| Phosphoric Acid (85%) | 30.150 | 33.500 | 30.150 | 33.500 |
| Total | | | | |
| % Weight reduction | 36.12 | 35.74 | 37.26 | 36.88 |

None of the above formulations were acceptable, however, because the inventors saw crystallization and thickening of the softgel fill solutions. Replacing copovidone or povidone K90 with povidone K12 also did not generate a suitable fill formulation; the solutions either crystallized upon mixing or formed a phase separation after 24 hours. Ultimately, the inventors discovered two softgel fill formulations containing phosphoric acid that provided the necessary characteristics. (See Example 1.)

### Example 9

Capsule Shell Formulations: The inventors also developed a shell capsule formulations that are effective for use with the concentrated fill formulations in some embodiments of the present disclosure, as shown below.

### Shell Formulation 1

| Component | % |
|---|---|
| Gelatin | 45.00 |
| Sorbitol sorbitan solution | 14.00 |
| (POLYSORB ® 85/70/00) | |
| Glycerin | 10.00 |
| FD&C Blue #1 | 0.08 |
| Purified water | 30.92 |
| Total | 100.00 |

### Shell Formulation 2

| Component | % |
|---|---|
| Gelatin | 45.00 |
| Sorbitol Special ™ Sorbitol | 14.00 |
| Sorbitan Solution | |
| Glycerin | 10.00 |
| FD&C Blue #1 | 0.08 |
| Purified water | 30.92 |
| Total | 100.00 |

### Example 10

Fill Formulation Manufacturing Protocol: Certain parameters were important in the manufacturing of the fill formulation, such as, the steps for adding the excipients and the naproxen sodium and maintaining certain temperature ranges during manufacturing. An example of an effective manufacturing protocol for Formula 1 is provided below.
Polyethylene glycol (Macrogol 400) is loaded into a reactor and stirred (anchor, 30 rpm) under nitrogen. A portion of total polyethylene glycol is reserved to drag the active pharmaceutical ingredient. The polyethylene glycol is heated to 40° C.-45° C., and copovidone is added to the reactor while stirring with anchor (30 rpm) once the temperature range is maintained.
The polyethylene glycol and copovidone are mixed by stirring (anchor, 30 rpm; dispersator 1500 rpm) for 30±5 min, under vacuum (<−0.7 bar), at The mixture is visually assessed for the complete dissolution of copovidone into the polyethylene glycol.
Upon dissolution, the temperature of the excipients is increased to 60-65° C. while stirring with anchor (30 rpm) under nitrogen.
Once the temperature is stabilized in the range, naproxen sodium is slowly added to the mixture, while mixing with a dispersator (1500 rpm). The mixture is maintained at a temperature of 60° C.-65° C. and continuously stirred (anchor, 30 rpm; dispersator 1500 rpm) for at least 60 minutes, under vacuum (≤−0.7 bar).

Phosphoric acid is then added to the mixture very slowly. During acid addition, the mixture is maintained at a temperature of 60° C.-65° C. and continuously stirred with a dispersator (1500 rpm). After acid addition, the mix is stirred (anchor, 30 rpm; dispersator 1500 rpm) at the prescribed temperature for at least 60 minutes, under nitrogen.

The formulation fill is cooled while stirring with anchor 20 rpm. When the blend reaches 45 ° C., vacuum is connected. The temperature of the final mixture is allowed to cool to 30 ±2° C. (≥60 minutes). At the end of the process the appearance of the suspension and the absence of air bubbles are checked.
The cooled mixture is stored in an enclosed tank (30 ±2° C., under nitrogen gas, in dark conditions) until encapsulation.

### Example 11

Provided below is an example showing an embodiment that includes a specific capsule formulation developed by the inventors and that contains the Formula 1 fill formulation of Example 1 and the shell composition of Example 8.

| Fill Material | mg/cps |
|---|---|
| Naproxen Sodium | 220.0 |
| Macrogol 400 NF/EP | 435.0 |
| COPOVIDONE | 20.0 |
| Phosphoric acid 85% | 33.5 |
| TOTAL | 708.5 |

| Shell Material | % |
|---|---|
| Gelatin | 45.00 |
| Sorbitol sorbitan solution | 14.00 |
| Glycerol | 10.00 |
| FD&C Blue # 1 | 0.080 |
| Purified water | 30.92 |
| TOTAL | 100.000 |

### Example 12

Provided below is an example showing another embodiment that includes a specific capsule formulation developed by the inventors and that contains the Formula 1 fill formulation of Example 1 and the shell composition of Example 8.

| Fill Material | mg/cps |
|---|---|
| Naproxen Sodium | 220.0 |
| Macrogol 400 NF/EP | 435.0 |
| COPOVIDONE | 20.0 |
| Phosphoric acid 85% | 33.5 |
| TOTAL | 708.5 |

| Shell Material | % |
|---|---|
| Gelatin | 45.00 |
| Sorbitol Special ™ Sorbitol | 14.00 |
| Sorbitan Solution | |
| Glycerol | 10.00 |
| FD&C Blue # 1 | 0.080 |
| Purified water | 30.92 |
| TOTAL | 100.000 |

The present disclosure is not to be limited in terms of the particular embodiments or implementations described in this application, which are intended as illustrations of various aspects. Many modifications and embodiments can be made without departing from its spirit and scope. Functionally equivalent methods and articles of manufacture within the scope of the disclosure, in addition to those enumerated herein, are possible from the foregoing descriptions. Such modifications and embodiments are intended to fall within the scope of the appended claims. The present disclosure is to be limited only by the terms of the appended claims, along with the full scope of equivalents to which such claims are entitled. This disclosure is not limited to particular methods, which can, of course, vary. The terminology used herein is for the purpose of describing particular embodiments only and is not intended to be limiting.

With respect to the use of substantially any plural and/or singular terms herein, the terms can be translated from the plural to the singular and/or from the singular to the plural as is appropriate to the context and/or application. The various singular/plural permutations may be expressly set forth herein for sake of clarity.

In general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

If a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having ordinary skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having ordinary skill in the art would understand the convention (e.g., “a system having at least one of A, B, or C” would include but not be limited to systems that have A alone, B alone, C alone, A and B together, A and C together, B and C together, and/or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, the disclosure is also thereby described in terms of any individual member or subgroup of members of the Markush group.

For any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. Language such as “up to,” “at least,” “greater than,” “less than,” and the like include the number recited and refer to ranges which can be subsequently broken down into subranges as discussed above. A range includes each individual member. Thus, for example, a group having 1-3 items refers to groups having 1, 2, or 3 items. Similarly, a group having 1-5 items refers to groups having 1, 2, 3, 4, or 5 items, and so forth.

While various aspects and embodiments have been disclosed herein, other aspects and embodiments are possible. The various aspects and embodiments disclosed herein are for purposes of illustration and are not intended to be limiting.

1. A pharmaceutical composition for oral administration comprising a soft gelatin capsule that encapsulates a liquid fill mixture, wherein the liquid fill mixture comprises:
(a) 30-32 wt. % naproxen salt;
(b) 60-62 wt. % polyethylene glycol;
(c) 1-4 wt. % copovidone;
(d) 4-6 wt. % phosphoric acid; and
wherein, the liquid fill mixture excludes glycerin and propylene glycol.

2. The composition of claim 1, wherein the soft gelatin capsule comprises:
(a) gelatin;
(b) sorbitol; and
(c) glycerin.

3. The composition of claim 2, wherein the gelatin capsule comprises:
a. purified water from about 28 to 32% by weight;
b. gelatin from about 40 to 50% by weight;
c. sorbitol from about 12 to 16% by weight;
d. glycerol from about 8 to 12% by weight;
e. colorants from about 0.05 to 1.2% by weight.

4. The composition of claim 3, wherein the gelatin capsule comprises:
a. purified water at about 30.9% by weight;
b. gelatin at about 45% by weight;
c. sorbitol at about 14% by weight;
d. glycerol at about 10% by weight;
e. colorants from about 0.1% by weight.

5. The composition of claim 1, wherein the naproxen salt is naproxen sodium.

6. (canceled)

7. The composition of claim 5, wherein
(a) the concentration of naproxen sodium is about 31.1% by weight;
(b) the concentration of polyethylene glycol is about 61.4% by weight;
(c) the concentration of phosphoric acid is about 4.7% by weight; and
(d) the concentration of copovidone is about 2.8% by weight.

8. The composition of claim 5, wherein the liquid fill mixture comprises:
(a) about 220 mg per capsule of naproxen sodium;
(b) about 435 mg per capsule of polyethylene glycol;
(c) about 20 mg per capsule of copovidone; and
(d) about 33.5 mg per capsule of phosphoric acid, wherein the phosphoric acid has concentration of 85%.

9. The composition of claim 1, wherein softgel capsules containing the composition are stable at room temperature for at least 24 months.

10. A method of making a liquid fill mixture for oral administration in a soft gelatin capsule, the method comprising:
(a) mixing polyethylene glycol and copovidone in a reaction chamber and heating to about 40-45° C.;
(b) increasing temperature of the mixture to about 60-65° C.; and
(c) adding naproxen salt and phosphoric acid while maintaining mixture temperature and stirring under vacuum.

11. A method of making a softgel capsule, using the method of claim 10, wherein the liquid fill mixture is encapsulated in softgel capsule shells.

12. The method of claim 10, wherein the softgel capsule shell prior to encapsulation has
a. a concentration of gelatin from about 40% to about 50% by weight;
b. a concentration of sorbitol from about 10% to about 18% by weight;
c. a concentration of glycerin from about 8% to about 12% by weight; and a concentration of FD&C Blue dye from about 0.020% to about 0.1% by weight.

13. The method of making the softgel capsule of claim 11, wherein the naproxen salt is naproxen sodium.

14. The method of making the softgel capsule of claim 13, wherein the liquid fill mixture comprises:
(a) naproxen sodium from about 30% to about 32% by weight;
(b) polyethylene glycol from about 60% to about 62% by weight;
(c) phosphoric acid from about 4% to about 6% by weight; and
(d) copovidone from about 1% to about 4% by weight.
